(19) United States
(12) Patent Application Publication
(10) Pub. No.: US 2016/0338681 A1
(43) Pub. Date: Nov. 24, 2016

(54) TISSUE RESECTION CAP WITH MECHANICAL TISSUE MANIPULATOR

(71) Applicant: Boston Scientific Scimed, Inc., Maple Grove, MN (US)

(72) Inventors: Paul SMITH, Smithfield, RI (US); Samuel RAYBIN, Marlborough,, MA (US); Jeff GRAY, Sudbury, MA (US); Celine CHIN, Brookline, MA (US); Matthew JAGELSKI, West Roxbury, MA (US)

(21) Appl. No.: 15/160,458

(22) Filed: May 20, 2016

Related U.S. Application Data
(60) Provisional application No. 62/164,918, filed on May 21, 2015.

(51) Int. Cl. A61B 17/00; A61B 1/00; A61B 1/06; A61B 1/05; A61B 17/32; A61B 1/018

(52) U.S. Cl. CPC A61B 17/00234; A61B 17/32002; A61B 1/018; A61B 1/0676; A61B 1/05; A61B 1/00089; A61B 2017/00269; A61B 2017/00353; A61B 2017/00296; A61B 2017/320032; A61B 1/00133

(57) ABSTRACT

The present disclosure relates to the field of endoscopy. In particular, the present disclosure relates to systems and methods for dissecting large areas of submucosal tissue. The system may apply tension to and continuously manipulate mucosal tissue such that large lesions may be dissected by a cutting element disposed at the distal end of an endoscope.

### FIELD

The present disclosure relates to the field of endoscopy. Specifically, the present disclosure relates to systems and methods for endoscopic submucosal dissection (ESD). More specifically, the present disclosure elates an endoscope hood that includes a tissue engaging element configured to apply tension to and continuously manipulate tissue such that large submucosal lesions may be dissected.

### BACKGROUND

Organ walls are composed of several layers: the mucosa (surface layer), the submucosa, the muscularis (muscle layer) and the serosa (connective tissue layer). A variety of lesions comprising dead, diseased or abnormal tissue may form on the mucosal walls of the colon, esophagus, stomach and duodenum. For example, gastrointestinal, colonic and esophageal cancers may form within the mucosal layer and manifest as a polyp or tissue mass that extends into the lumen of the respective organ. Endoscopic mucosal resection (EMR) is a minimally invasive technique by which such lesions may be removed without disrupting the integrity of the organ wall. EMR is generally performed using an endoscope that includes a long narrow tube equipped with a light, video camera and one or more channels to receive a variety of medical instruments. The endoscope is passed down the esophagus or guided through the rectum to the site of the target lesion. The distal end of the endoscope is further equipped with an endoscope hood (i.e., cap) that is positioned over the tissue to be resected. Once properly positioned, the lesion is resected using a variety of techniques known in the art.

State-of-the-art endoscope hoods are appropriate for EMR procedures because they provide a working volume that is approximately the size of the lesion to be resected. Unfortunately, lesions within the submucosal tissue layer tend to be significantly larger than mucosal lesions and cannot be removed using a standard EMR hood. Due to the larger size of submucosal lesions, endoscopic submucosal dissection (ESD) procedures require an endoscope hood that provides greater visualization of the working area and the ability to manipulate the mucosal tissue to expose/dissect the entire lesion.

### SUMMARY

The present disclosure, in its various aspects, meets an ongoing need in the field of endoscopy for safe and efficient removal of submucosal lesions. The present disclosure provides a high-visibility endoscope hood that allows the medical professional to manipulate mucosal tissue such that the entire submucosal lesion is exposed for removal.

In one aspect, the present disclosure relates to a medical device, comprising: a substantially circular endoscope hood that includes a proximal end and a distal end, wherein the distal end defines an opening; a tissue engaging element coupled to the distal end of the endoscope hood, wherein the tissue engaging element is moveable relative to the opening of the endoscope hood; and an actuator configured to move the tissue engaging element. The tissue engaging element may be moveable in at least one of an axial, radial, rotational and/or transverse manner relative to the plane defined by the opening of the endoscope hood. The actuator may move the tissue engaging element in a longitudinal, rotational and or vertical direction relative to the plane defined by the opening of the endoscope hood. The tissue engaging element may include a substantially cylindrical member that rotates an axis that is perpendicular to a longitudinal axis of the endoscope. The tissue engaging element may also include a substantially planar member that rotates about its longitudinal axis. The tissue engaging element may include a substantially spherical member that rotates about plurality of axes. The tissue engaging element may include an arm that is moveable between a constrained configuration and an unconstrained configuration. The tissue engaging element may be an extension of the actuator that forms a loop that includes a generally upward curve. A surface of the tissue gripping element include a surface feature, including, for example, at least one outwardly extending projection configured to couple a layer of tissue to the surface of the gripping element. The at least one outwardly extending projection may include a plurality of ribs and grooves. The at least one outwardly extending projection may include a plurality of knobs, teeth or fingers. The tissue gripping elements configured to restrict relative movement between the layer of tissue and the distal end of the endoscope hood. The tissue engaging element may move in a direction towards the distal opening of the endoscope hood. Alternatively, the tissue engaging element may move in a direction away from the distal opening of the endoscope hood. The actuator may extend along a length of the endoscope hood between the proximal and distal ends. The actuator may extend along an outer wall of the endoscope hood. The actuator may extend along an inner wall of the endoscope hood. The actuator may include a cable, an elongate shaft or hydraulic tubing. Extension and/or retraction of the cable or elongate shaft may cause the tissue engaging element to move. Alternatively, rotation of the cable or elongate shaft may cause the tissue engaging element to move. Pressure applied to a fluid within the hydraulic tubing may likewise cause the tissue engaging element to move. The actuator may be connected to a power source located within, or external to, the endoscope hood.

In another aspect, the present disclosure relates to a medical device comprising an endoscope that includes a proximal end, a distal end and at least one working channel extending therebetween; a substantially circular endoscope hood that includes a proximal end and a distal end, wherein the distal end defines an opening; a tissue engaging element coupled to the distal end of the endoscope hood, wherein the tissue engaging element is moveable relative to the opening of the endoscope hood; and an actuator configured to move the tissue engaging element. A cutting element may be slidably and/or rotationally disposed within the at least one working channel of the endoscope and extending at least partially into the distal opening of the endoscope hood. The distal end of the endoscope may include a light source configured to illuminate the distal opening of the endoscope hood. The distal end of the endoscope may also include a camera to visualize the distal opening of the endoscope hood.

In yet another aspect, the present disclosure relates to a method, comprising: disposing within the body of a patient a medical device comprising a substantially circular endoscope hood, wherein the endoscope hood includes: a proximal end and a distal end, wherein the distal end defines an opening; a tissue engaging element coupled to the distal end of the endoscope hood, wherein the tissue engaging element is moveable relative to the opening of the endoscope hood; and an actuator configured to move the tissue engaging element; and activating the tissue engaging element to move a tissue within the body of the patient. Activating the tissue engaging element may move the tissue in a direction towards the distal opening of the endoscope hood. Alternatively, activating the tissue engaging element may move the tissue in a direction away from the distal opening of the endoscope hood. Extension and/or retraction of the actuator may also cause the tissue engaging element to move. Rotation of the actuator may likewise cause the tissue engaging element to move.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 depicts the distal end of an endoscope attached to an endoscope hood that includes a cylindrical rotatable tissue engaging element with a series of ribs and grooves for gripping tissue, in accordance with one embodiment of the present disclosure.

FIG. 2 depicts the distal end of an endoscope attached to an endoscope hood that includes a cylindrical rotatable tissue engaging element with a series of finger-like projections for gripping tissue, in accordance with another embodiment of the present disclosure.

FIG. 3 depicts the distal end of an endoscope attached to an endoscope hood that includes a rotary belt tissue engaging element with a series of teeth for gripping tissue, in accordance with another embodiment of the present disclosure.

FIG. 4 depicts the distal end of an endoscope attached to an endoscope hood that includes a spherical rotatable tissue engaging element with a series of knobs for gripping tissue, in accordance with another embodiment of the present disclosure.

FIG. 5 depicts the endoscope hood of FIG. 1, in which the cylindrical rotatable tissue engaging element is disposed on an extendable, retractable and/or rotatable post, in accordance with another embodiment of the present disclosure.

FIGS. 6A-B depict the distal end of an endoscope attached to an endoscope hood that includes a longitudinally movable tissue engaging element in the retracted (FIG. 6A) and extended (FIG. 6B) configurations, in accordance with another embodiment of the present disclosure.

FIGS. 7A-B depict the distal end of an endoscope attached to an endoscope hood that includes a tissue engaging element moveable between a constrained (i.e., linear) configuration and an unconstrained (i.e., elevated) configuration, in accordance with another embodiment of the present disclosure.

FIG. 8 depicts the distal end of an endoscope attached to an endoscope hood that includes a fixed (i.e., immovable) tissue engaging element, wherein the endoscope hood rotates about the distal end of the endoscope, in accordance with another embodiment of the present disclosure.

FIG. 9 depicts the distal end of an endoscope attached to an endoscope hood that includes a rotatable tissue engaging element, wherein the endoscope hood also rotates about the distal end of the endoscope, in accordance with another embodiment of the present disclosure.

FIG. 10 depicts a tissue engaging element that includes an extendable/retractable curved bar disposed within an endoscope hood, in accordance with another embodiment of the present disclosure.

FIGS. 11A-D depict the extendable/retractable curved bar disposed within (FIGS. 11A-B) and extended outside (FIGS. 11C-D) the endoscope hood, in accordance with another embodiment of the present disclosure.

It is noted that the drawings are intended to depict only typical or exemplary embodiments of the disclosure. It is further noted that the drawings may not be necessarily to scale. Accordingly, the drawings should not be considered as limiting the scope of the disclosure. The disclosure will now be described in greater detail with reference to the accompanying drawings.

### DETAILED DESCRIPTION OF THE PREFERRED EMBODIMENTS

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to an endoscope hood attached to the distal end of an endoscope, it should be appreciated that the endoscope hood disclosed herein may be attached to a variety of medical devices that are inserted into a lumen of a patient, including for example, guide lumens, ports, optical wands and the like. As used herein, the term “distal” refers to the end farthest away from a medical professional when introducing a device into a patient, while the term “proximal” refers to the end closest to the medical professional when introducing a device into a patient.

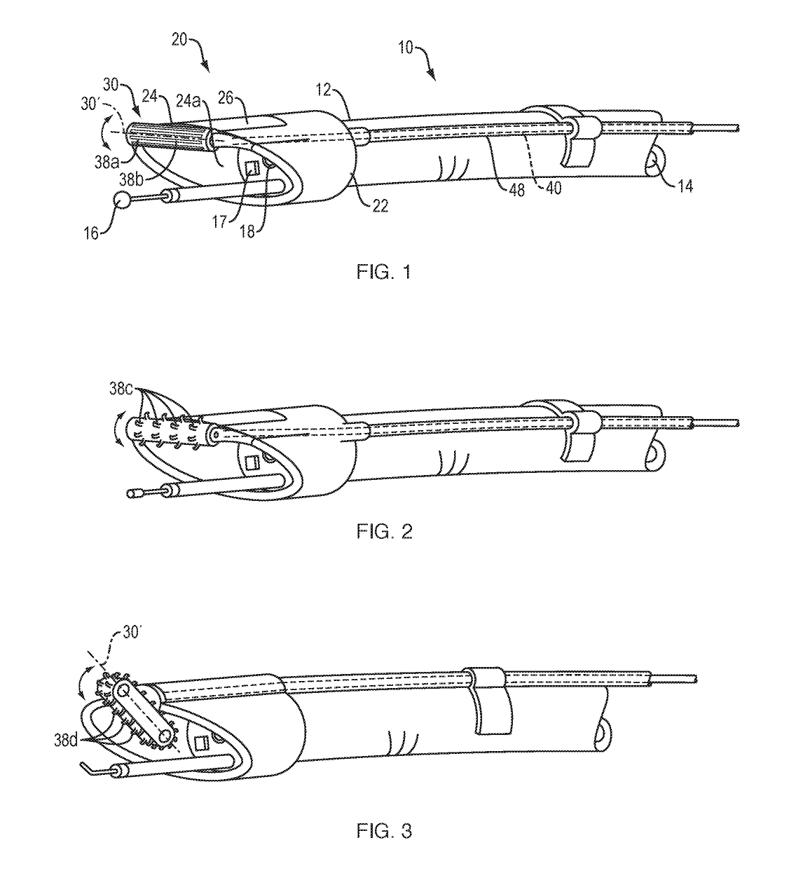

In one embodiment, the present disclosure provides a system for dissecting submucosal tissue. As illustrated in FIG. 1, the system generally comprises an endoscope hood 20 that includes a proximal end 22, a distal end 24 defining an opening 24a, a tissue engaging element 30 coupled to the distal end 24 and an actuator 40 configured to move the tissue engaging element 30. The proximal end 22 includes an opening (not shown) configured to receive the distal end 12 of an endoscope 10. The endoscope 10 defines at least one working channel 14 through which a variety of medical instruments may extend. The distal end of the endoscope 10 may further include a light source 17 and camera 18 for visualizing the mucosal tissue as well as any medical instruments, including, for example, the cutting tool 16.

The endoscope hood 20 includes a substantially cylindrical shape that allows narrow body lumens and/or cavities to be navigated in as minimally invasive a manner as possible. The opening 24a of the endoscope hood 20 defines a plane that slants/slopes at an oblique angle such that one side (i.e., the top side) of the distal end 24 extends farther than the opposite side (i.e., the bottom side). The oblique shape of the opening 24a increases the effective surface area of the opening to provide an optically clear path that allows the medical professional to more readily identify the size and shape of the submucosal lesion. The increased surface area of the opening 24a also provides a larger working channel that allows the medical professional to more efficiently and accurately access and clear adjoining mucosal tissue during the ESD procedure. In one embodiment, optimal tissue dissection is achieved with an endoscope hood that includes an oblique opening with an angle of at least 30 degrees, more preferably at least 45 degrees and even more preferably at least 60 degrees.

The oblique shape of the distal end 24 of the endoscope hood 20 also serve as a reference point that enhances the medical professional's depth perception, further facilitating accurate positioning of the tissue engaging element 30 and cutting element 16 for precise tissue manipulation and submucosal lesion dissection. To this end, an inner surface of the distal end 24 of the endoscope hood 20 may include horizontal and/or vertical demarcations (i.e., lines, etchings, ribs etc.) (not shown) that provide the medical professional with a rough approximation of the size of objects viewed through the endoscope, and relative distance between such objects. The ability to gauge size and distance further enhances the medical professional's ability to manipulate the endoscope and attached medical instruments to more precisely control the dissection of diseased tissue from healthy tissue. The horizontally and/or vertically disposed ribs may also pro Tide additional structural support to the endoscope hood, thereby allowing the wall thickness of the endoscope hood to be thinner and less invasive when inserted into a patient. The endoscope hood 20 may also include one or more holes/vents 26 to permit the flow of air to dissipate smoke resulting from the electrosurgical cutting. This, again, further enhances the medical professional's ability to visualize the tissue being dissected.

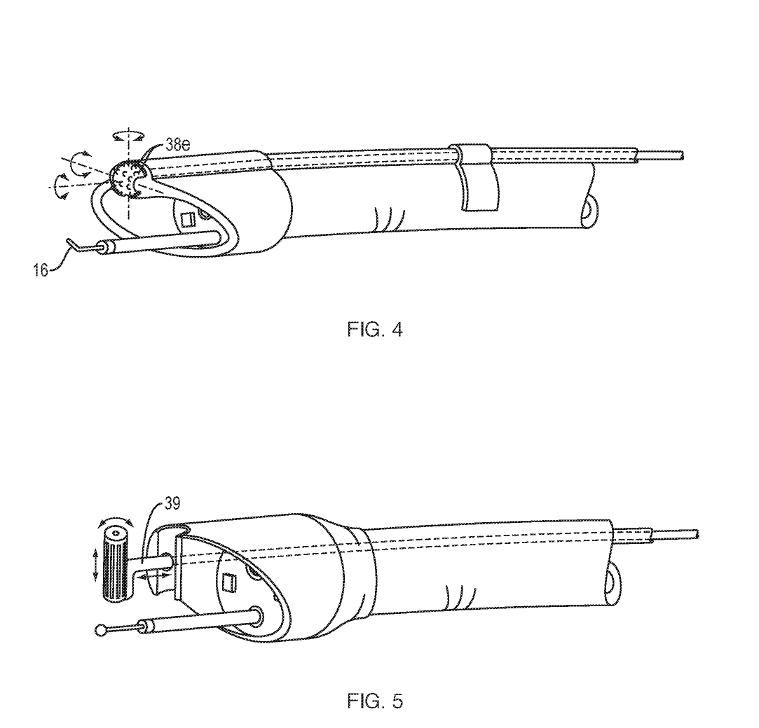

In one embodiment, the tissue engaging element 30 includes a rotatable member configured to move rotationally about a point, an axis or plurality of axes. The surface of the rotatable member may comprise a geometry, surface characterization and/or material configured to grasp, grip or otherwise engage a layer of mucosal tissue. For example, the surface of the rotatable member may include one or more gripping elements configured to grip the mucosal tissue as the rotatable member rotates, thereby coupling a layer of mucosal tissue to the surface of the rotatable member. Non-limiting examples of gripping elements may include an alternating series of ribs 38a and groove 38b (FIG. 1), finger-like projections 38c (FIG. 2), teeth 38d (FIG. 3) and/or knobs 38e (FIG. 4).

Depending on the direction of rotation, the rotatable member may pull the mucosal tissue towards the oblique opening 24a, or push the mucosal tissue away from the oblique opening. The dexterity afforded by the rotatable member allows the medical professional to manipulate the mucosal tissue layer to expose new working area and better identify the delineation between the submucosal lesion and surrounding healthy tissue. Once the submucosal lesion (or a portion thereof) has been identified, the rotatable member may be used to maintain the mucosal tissue in a stretched configuration (i.e., by pushing the tissue away from the oblique opening) so that the cutting element 16 may be precisely positioned. The rotatable member may also be used to draw the submucosal lesion towards the cutting element by pulling the tissue towards the oblique opening.

Movement of the rotatable member is controlled by an actuator 40 disposed within a housing 48 that extends along the length of the endoscope hood 20 and endoscope 10. As illustrated in FIGS. 1-4, the housing 48 and actuator 40 may extend along an outer wall of the endoscope hood 20 and endoscope 10. Alternatively, the housing 48 and actuator 40 may extend along an inner wall of the endoscope hood 10 and endoscope 10 (not shown).

In one embodiment, the actuator includes an elongate shaft (i.e., drive shaft) configured to transfer rotational motion to the rotatable member. For example, the distal end of the elongate shaft may include a gear (pinion, liner “worm” etc.) configured to engage a corresponding gear attached to the rotatable member, such that rotation of the elongate member is transferred to the rotatable member. The direction of rotation of the rotatable member may be controlled by changing the direction of rotation (i.e., clockwise or counter-clockwise) of the elongate shaft. In another embodiment, rotation of the elongate shaft may be provided by a direct drive rotary (DDR) motor configured to drive rotation of the rotatable member without the need for gears, belts, pulleys or chains.

In another embodiment, the elongate shaft may be configured to move longitudinally to transfer motion to rotatable member. For example, an over-running clutch located within, or adjacent to, the rotatable member could impart front-to-back (i.e., proximal-to-distal) movement of the elongate shaft into rotational movement.

In yet another embodiment, the actuator may include a cable that forms a loop extending the length of the housing 48. The cable includes a middle portion that wraps around a gear attached to the rotatable member, and free ends that extend beyond the proximal end of the endoscope. Pulling on one end of the cable would cause the rotatable member to rotate in one direction, while pulling the other end of the cable would cause the rotatable member to rotate in the opposite direction. In yet another embodiment, the cable may be configured to push or pull a rack gear attached to the rotatable member.

Rotation of the rotatable member may be hand-powered, including, for example by a thumbwheel located on a handle attached to a proximal end of the endoscope. The medical professional may, for example, advance and/or retract the thumbwheel as necessary to impart rotation (clockwise or counter-clockwise) to the rotatable member. Alternatively, the rotation of the rotatable member may be controlled by an external power supply connected to the actuator 40. For example, the power supply may be an external electric motor located on the handle of the endoscope. The power supply may also be a self-contained micro-motor located on or within the endoscope hood that is controlled wirelessly. The power supply could be coupled to a user interface or driving means such as a servo.

FIG. 1 illustrates one embodiment of an ESD system, in which the tissue engaging element 30 includes a cylindrical rotatable member configured to rotate about an axis 30′, which is perpendicular to a longitudinal axis of the endoscope 10. The cylindrical rotatable member includes an alternating series of ribs 38a and grooves 38b for gripping tissue. FIG. 2 illustrates another embodiment of an ESD system, in which the cylindrical rotatable member includes a plurality of outwardly projecting fingers 38c (i.e., finger-like projections) for gripping tissue. FIG. 3 illustrates another embodiment of an ESD system, in which the tissue engaging element 30 includes a rotary belt disposed over two or more parallel rotating elements. The planar surface of the rotary belt optionally includes a plurality of teeth 38d for gripping tissue. FIG. 4 illustrates yet another embodiment of an ESD system, in which the tissue engaging element 30 includes a substantially spherical rotatable member (i.e. a ball-head) configured to rotate about a plurality of axes (i.e., 360°). The surface of the substantially spherical rotatable member may include a plurality of knobs 38e for gripping tissue. While the ball-head may rotate in any direction, strict radially upward motion is not required to draw tissue away from the cutting element 16, as long as the motion is generally upward. Similarly, strict radially down motion is not required to draw tissue towards the cutting element 16, as long as the motion is generally downward. In one embodiment, the actuator 40 imparts rotational movement to the substantially spherical rotatable member by engaging a ball-head (not shown) located within the distal end of the endoscope hood.

FIG. 5 illustrates an embodiment of FIG. 1, in which the rotatable member is attached the distal end of the endoscope hood by a post 39. As indicated by the direction of the arrows, the post 39 is configured to extend, retract and rotate independent of the endoscope hood and endoscope such that the rotatable member may be oriented in any direction required by the medical professional to manipulate the tissue. It should be appreciated that the post 39 is not limited to the rotatable member depicted in FIG. 1, but may be used with any of the tissue engaging elements disclosed herein.

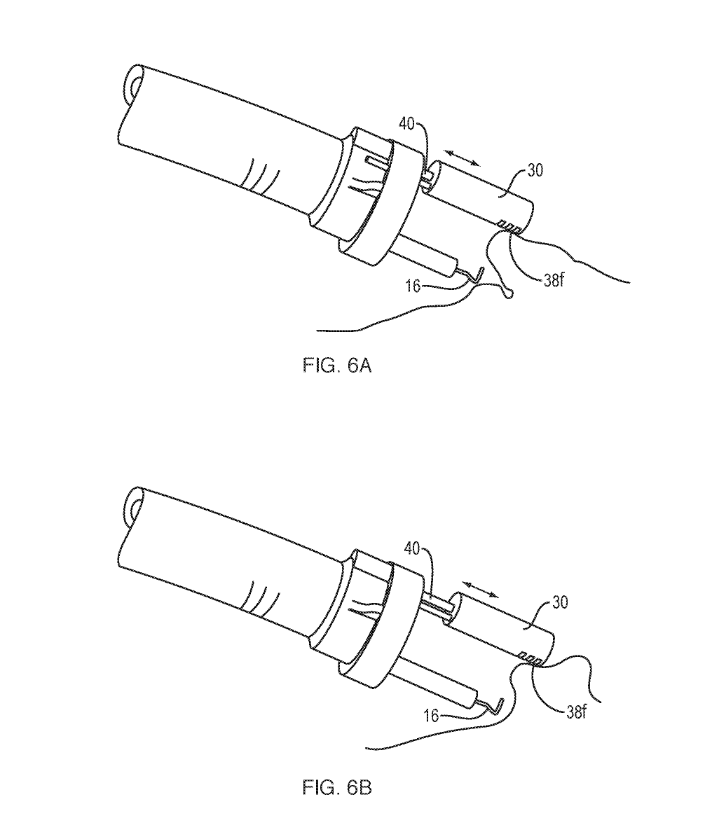

The present disclosure is in no way limited to tissue engaging elements that are rotatable. For example, FIGS. 6A-6B illustrate an embodiment of an ESD system, in the tissue engaging element 30 is configured to extend and/or retract (i.e., move distally and/or proximally) relative to the distal end of the endoscope hood. The tissue engaging element 30 includes an irregular (i.e., course or jagged) surface 38f configured to grip the mucosal tissue as the tissue engaging element 30 extends and/or retracts. As above, the dexterity afforded by the extendable/retractable tissue engaging element allows the medical professional to manipulate the mucosal tissue layer to expose new working area and better identify the delineation between the submucosal lesion and surrounding healthy tissue. Once the submucosal lesion (or a portion thereof) has been identified, the extendable/retractable tissue engaging element may be used to maintain the mucosal tissue in a stretched configuration so that the cutting element 16 may be precisely positioned. Alternatively, the tissue engaging element may be used to draw the submucosal lesion towards the cutting element 16. Movement of the extendable/retractable tissue engaging element may b controlled by an actuator 40 that includes, for example, a push-pull cable or pneumatic/hydraulic tubing.

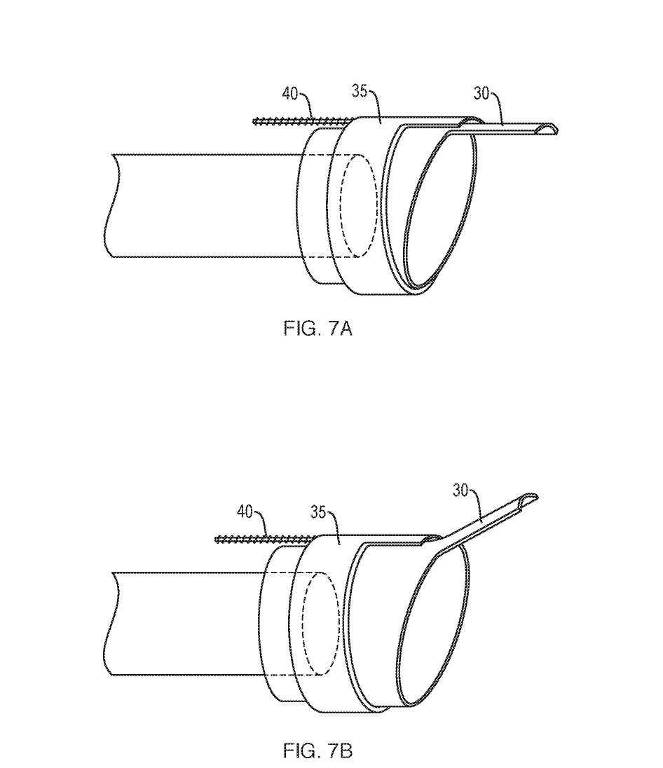

FIGS. 7A-7B illustrate another embodiment of an ESD system, in which the tissue engaging element 30 includes a rigid/inflexible arm configured to move from a constrained configuration (FIG. 7A) to a relaxed configuration (FIG. 7B). The medical professional controls the deflection of the arm using a sleeve 35 slidably disposed about the surface of the endoscope hood, which forces the rigid/inflexible arm into the constrained configuration. When in the constrained configuration the arm is positioned underneath a portion of the mucosal tissue. Once properly positioned, the sleeve is retracted proximally (withdrawn), thereby allowing the rigid/inflexible arm to deflect to the unconstrained configuration (i.e., deflected upward) to lift the tissue, and expose a desired location for cutting. Movement of the extendable/retractable tissue engaging element may be controlled by an actuator 40 that includes, for example, a push-pull cable or pneumatic/hydraulic tubing.

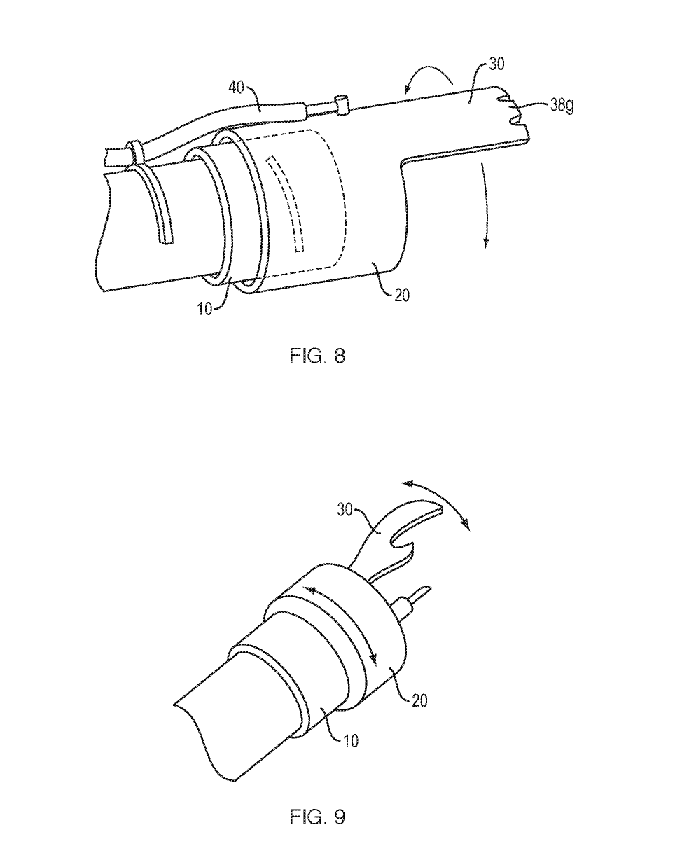

FIG. 8 illustrates another embodiment of an ESD system, in which the tissue engaging element 30 includes a fixed arm with an irregular end 38g attached to an endoscope hood 20 that rotates about the distal end of the endoscope 10. FIG. 9 illustrates another embodiment of an ESD system, in which the tissue engaging element 30 includes a rotatable grasper attached to an endoscope hood 20 that rotates about the distal end of the endoscope 10. As will be understood by those in the art, the ability of the endoscope hood to rotate about the distal end of the endoscope provides the medical professional with still further dexterity in controlling the location of the tissue engaging element (fixed or moveable) and cutting element 16.

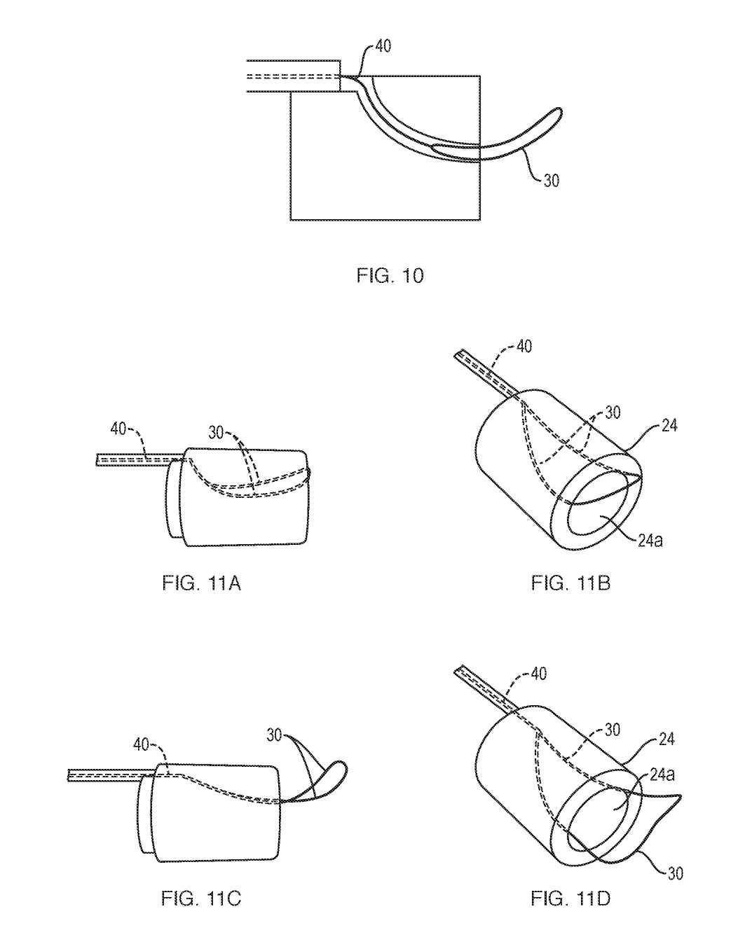

FIG. 10 illustrates yet another embodiment of an ESD system, in which the tissue engaging element 30 includes an extendable and/or retractable curved bar. As best shown in FIGS. 11A-11B the curved bar is disposed within the opening 24a at the distal end 24 of the endoscope hood to facilitate smooth travel through the working channel of the endoscope and minimally invasive positioning within the patient, and to provide a clearer view of the working space. Alternatively, the curved bar may he disposed between an inner and outer cap (not shown). Movement of the curved bar may be controlled by an actuator 40 that includes, for example, a push-pull cable or pneumatic/hydraulic tubing. Once the endoscope hood is positioned in the vicinity of a mucosal tissue layer that requires manipulation, the actuator is extended to push the curved bar out of the endoscope hood (FIGS. 11C-11D). Due to its curvilinear shape, the curved bar exits the endoscope hood at an upward angle that contacts and lifts the mucosal tissue layer. Although the curved bar depicted in FIGS. 11A-D is formed from a continuous section of wire, it should be appreciated that the curved bar may include a variety of shapes, including, for example, a solid piece of metal or plastic.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
a substantially circular endoscope hood that includes a proximal end and a distal end, wherein the distal end defines an opening;
a tissue engaging element coupled to the distal end of the endoscope hood, wherein the tissue engaging element is moveable relative to the opening of the endoscope hood; and
an actuator configured to move the tissue engaging element.

2. The medical device of claim 1, wherein the tissue engaging element is moveable in at least one of an axial, radial, rotational and/or transverse manner relative to the plane defined by the opening of the endoscope hood.

3. The medical device of claim 1, wherein the actuator is configured to move the tissue engaging element in a longitudinal, rotational or vertical direction relative to the plane defined by the opening of the endoscope hood.

4. The medical device of claim 1, wherein the tissue engaging element is a substantially cylindrical member that is configured to rotate about an axis that is perpendicular to a longitudinal axis of the endoscope.

5. The medical device of claim 1, wherein the tissue engaging element includes a substantially planar member configured to rotate about a longitudinal axis of the device.

6. The medical device of claim 1, wherein the tissue engaging element is a substantially spherical member that rotates about plurality of axes.

7. The medical device of claim 1, wherein the tissue engaging element includes an arm that is moveable between a constrained configuration and an unconstrained configuration.

8. The medical device of claim 1, wherein the tissue engaging element is an extension of the actuator that forms a loop.

9. The medical device of claim 1, wherein the tissue engaging element moves in a direction towards the distal opening of the endoscope hood.

10. The medical device of claim 1, wherein the tissue engaging element moves in a direction away from the distal opening of the endoscope hood.

11. The medical device of claim 1, wherein the actuator is a cable.

12. The medical device of claim 1, wherein the actuator is an elongate shaft.

13. The medical device of claim 1, wherein the actuator is hydraulic tubing.

14. A medical device, comprising:
an endoscope that includes a proximal end, a distal end and at least one working channel extending therebetween;
a substantially circular endoscope hood attached to the distal end of the endoscope, wherein the endoscope hood includes:
a proximal end and a distal end, wherein the distal end defines an opening;
a tissue engaging element coupled to the distal end of the endoscope hood, wherein the tissue engaging element is moveable relative to the opening of the endoscope hood; and
an actuator configured to move the tissue engaging element.

15. The medical device of claim 14, further comprising a cutting element disposed within the at least one working channel of the endoscope, wherein the cutting element is slidably and rotationally disposed within the at least one working channel of the endoscope.

16. A method, comprising:
disposing within the body of a patient a medical device comprising a substantially circular endoscope hood, wherein the endoscope hood includes:
a proximal end and a distal end, wherein the distal end defines an opening;
a tissue engaging element coupled to the distal end of the endoscope hood, wherein the tissue engaging element is moveable relative to the opening of the endoscope hood; and
an actuator configured to move the tissue engaging element; and
activating the tissue engaging element to move a tissue within the body of the patient.

17. The method of claim 16, wherein activating the tissue engaging element moves the tissue in a direction towards the distal opening of the endoscope hood.

18. The method of claim 16, wherein activating the tissue engaging element moves the tissue in a direction away from the distal opening of the endoscope hood.

19. The method of claim 16, wherein extension and/or retraction of the actuator causes the tissue engaging element to move.

20. The method of claim 16, wherein rotation of the actuator causes the tissue engaging element to move.
